(19) United States
(12) Patent Application Publication
(10) Pub. No.: US 2011/0171296 A1
(43) Pub. Date: Jul. 14, 2011

(54) METHOD AND PREPARATION FOR BINDING ACETALDEHYDE IN SALIVA, THE STOMACH AND THE LARGE INTESTINE

(75) Inventors: Mikko Salaspuro, Helsinki (FI); Martti Marvola, Helsinki (FI)

(73) Assignee: Biohit Oyj, Helsinki (FI)

(21) Appl. No.: 13/004,302

(22) Filed: Jan. 11, 2011

Related U.S. Application Data
(63) Continuation-in-part of application No. 10/415,422, filed on Nov. 21, 2003.

(30) Foreign Application Priority Data
Oct. 30, 2000 (FI) 20002392

(51) Int. Cl. A61K 31/198; C07K 14/435; A61K 9/28; A61K 9/52; C07C 321/04; A61P 35/00

(52) U.S. Cl. 424/452; 530/350; 424/474; 514/562; 562/557

(57) ABSTRACT

The object of the invention is the use of compounds comprising one or more free sulphhydryl or amino groups for preparing a pharmaceutical composition for locally binding acetaldehyde in saliva, the stomach or the large intestine, and pharmaceutical compositions comprising the said compounds.

### RELATED APPLICATIONS

This application is a continuation-in-part application of U.S. application Ser. No. 10/415,422, filed Nov. 21, 2003, which is a §371 national stage application of International App. No. PCT/FI01/00948 filed Oct. 30, 2001, which claims benefit of priority to Finnish Application No. FI 20002392 filed October 2000, the entire contents of which are hereby incorporated by reference.

The object of the invention is the use according to the preamble of claim 1 for preparing a pharmaceutical composition for locally binding the acetaldehyde in saliva, the stomach, and the large intestine. Another object of the invention is the pharmaceutical compositions according to the preambles of claims 6, 11, and 19 and a method according to the preamble of claim 28 for decreasing the risk of developing cancer of the mouth, the pharynx, the oesophagus, the stomach, or the large intestine.

It has been found that acetaldehyde causes cancer in animals (Feron et al, (1982) Eur J Cancer Clin Oncol 18:13-31). It has also been found that acetaldehyde is a local carcinogen, when occurring in human saliva and the alimentary tract. This is supported by the fact that Asian heavy consumers of alcohol, who have a familial low-activity modification of aldehyde dehydrogenase-2 (ALDH2) enzyme, have both an increased risk of developing cancer of the mouth, the pharynx, and the alimentary tract, and an increased acetaldehyde content of the saliva after consuming alcohol (Väkeväinen et al, (2000) Alcohol Clin Exp Res 24:873-877).

In the organism, acetaldehyde is formed from alcohol as a consequence of hepatic metabolism and, according to recent research, locally in the alimentary tract through a microbial alcohol dehydrogenase (Salaspuro et al, (1996) Ann Med 28:195-200).

After moderate consumption of ethanol, for example, high acetaldehyde contents of a microbial origin have been found in human saliva; in other words, acetaldehyde builds up in saliva as an intermediate product of microbial metabolism (Homann et al, Carcinogenesis (1997) 18:1739-1743).

Acetaldehyde also builds up in the stomach as a consequence of microbial metabolism in a situation, where there is no acid in the stomach or the acid has been removed by medication (Väkeväinen et al, (2000) Alimentary Pharmacology Therapeutics, in press). It has also been shown that acetaldehyde builds up in the large intestine, because its bacteria that represent the normal flora are capable of converting ethanol into acetaldehyde (Jokelainen et al, (1996) Gut 39:100-104).

The average amount of saliva excreted by a human is 1.5 litres a day. The areas of influence of the acetaldehyde contained by the saliva include the mouth, the pharynx, the oesophagus, and the stomach.

The highest acetaldehyde contents of the organism in connection with consuming alcohol occur in the contents of the large intestine and in the saliva.

Endogenous ethanol, i.e., ethanol that builds up in the intestines in oxygen-free conditions as a consequence of microbes can also be found in the intestines. When this ethanol gets into contact with oxygen near the mucous membrane, for example, acetaldehyde is formed.

Acetaldehyde is also formed in the mouth, the pharynx, and the upper airways as a consequence of smoking and exposure to air contamination. It has been proven that chronic smoking increases the acetaldehyde production of saliva originated in microbes.

Pharmaceutical compositions containing the effective substances according to the present invention are known from before, the alleged effect of which is based on the reaction of the effective substances to the acetaldehyde inside blood and/or cells. On the basis of prior art publications it is doubtful whether the preparations in question are even capable of decreasing the acetaldehyde content of blood originating in alcohol. The known preparations also have such a composition that they would not be able to bind, on a long-term basis, the acetaldehyde, which is locally generated in the organism and which locally occurs in high contents (see publications U.S. Pat. No. 5,202,354, U.S. Pat. No. 4,496,548, U.S. Pat. No. 4,528,295, U.S. Pat. No. 5,922,346).

The acetaldehyde, which forms in the organism during the consumption of alcohol and afterwards, also causes physiological symptoms called a hangover. Previously, an effort has been made to decrease the symptoms caused by acetaldehyde by taking preparations containing ascorbic acid, thiamine, cysteine or cysteic acid, and flavonoids or flavonoid complexes in a form of tablets swallowed orally in connection with, before or after consuming alcohol. It is believed that the method in question mostly decreases the acetaldehyde content in blood, because when swallowed, the effective substances go to the stomach and from there into the blood circulation. The tablets used in the method contained small amounts of effective substances only, and therefore had no effect on the acetaldehyde in saliva or the stomach (Matsuoka, U.S. Pat. No. 5,202,354 and Moldowan et al, U.S. Pat. No. 4,496,548).

It has been suggested that preparations containing amino acids, such as L-cysteine, methionine, taurine or arginine, ascorbic acid and vitamins A and E, which are sucked or chewed in the mouth be used to decrease the effect of the harmful free radical compounds that are formed in connection with using tobacco products or being exposed to them. It is believed that amino acids affect various tissues after being absorbed (Hersch, U.S. Pat. No. 5,922,346, Hersch, International Patent Application No PCT/US98/12617). However, the number of substances capable of binding the acetaldehyde and contained by these preparations is very low, the effect very short-term; hence, we are not talking about a local long-term effect.

So far, neither a method nor a preparation has been presented, which would locally decrease the acetaldehyde content of saliva, the mouth, the pharynx, the stomach or the large intestine. The methods and preparations according to prior art contain acet-aldehyde-binding substances in small amounts only, or their impact is very short-term, whereby the content of acetaldehyde quickly regains its previous level after the effect of the substances has ended.

The object of the invention is to provide a method and a preparation for decreasing or removing the acetaldehyde content of the saliva, and consequently, that of the pharynx, the oesophagus, the stomach, and separately that of the large intestine and the stomach from the area of the mouth and the alimentary tract and from the upper airways. The use, the composition, and the method according to the invention are very useful in locally binding the increased acetaldehyde that occurs in connection with consuming alcoholic drinks or smoking. In principle, the acetaldehyde can originate from any source, such as a foodstuff containing acetaldehyde; the acetaldehyde can have been formed from the ethanol contained by the foodstuff or it can have been formed from an endogenous ethanol occurring in the organism. The purpose of the invention is to decrease the risk of contracting cancers of the mouth and the alimentary tract, which are caused by the acetaldehyde in the said areas.

The invention is based on the surprising observation that the harmful amount of acetaldehyde locally occurring in saliva, the stomach or the large intestine can be bound locally, quickly and in large concentrations into a chemically safe form by using the preparations according to the present invention. As the substances that bind it are released in contents high enough throughout the entire period of effect of the acet-aldehyde, the local acetaldehyde content remains low. In this way, the local risk of contracting cancer caused by acetaldehyde decreases.

To be more precise, the use according to the invention is characterized by what is stated in the characterizing part of claim 1.

According to the invention, compounds that comprise one or more free sulphhydryl and/or amino groups are used to prepare a pharmaceutical compound, which is used to locally bind the acetaldehyde in saliva, the stomach or the large intestine.

Other objects of the invention are a pharmaceutical composition according to the characterizing part of claim 6 for binding acetaldehyde from saliva, a pharmaceutical composition according to the characterizing part of claim 11 for binding acetaldehyde from the stomach, and a pharmaceutical composition according to the characterizing part of claim 19 for binding acetaldehyde from the large intestine.

According to the invention, the pharmaceutical composition comprises one or more substances that bind acetaldehyde, as bound to a pharmaceutically acceptable carrier. The substances contained by the composition are selected so that the substances that are capable of binding acetaldehyde are released within a long period of time.

Another object of the invention is a method according to the characterizing part of claim 28 for decreasing the effect of acetaldehyde, which causes cancer, in human saliva, the stomach or the large intestine.

According to the method, the acetaldehyde contained by saliva, the stomach or the large intestine is locally bound into a safe form by using a pharmaceutical composition that releases one or more acetaldehyde-binding substances.

The invention provides considerable advantages. The pharmaceutical compositions comprising acetaldehyde-binding compounds can be used to decrease the risk of developing cancer of the mouth, the pharynx, the oesophagus, the stomach, and the large intestine. In particular, the compositions according to the invention can be used for large-scale consumers of alcohol and especially for those, who have a familial low-activity modification of the aldehyde dehydrogenase-2 (ALDH2) enzyme. The use of the compositions according to the invention is also of benefit to those who consume moderate amounts of alcohol or who consume foodstuffs that contain small contents of alcohol or acetaldehyde. Furthermore, the use of the compositions according to the invention also benefits smokers.

In the following, the present invention is examined more closely with the aid of a detailed description and examples. The appended drawing shows the measuring results of the acetaldehyde contents of the saliva of the test groups as the function of time, according to Example 1.

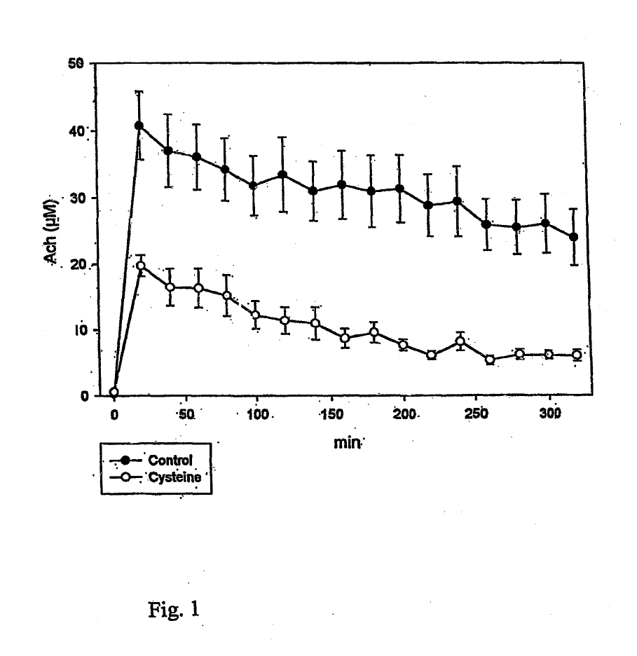

FIG. 1 shows the acetaldehyde content as the function of time in the saliva of the persons in a control group and that of the participants in the test group who used the acetaldehyde-binding preparation according to the invention.

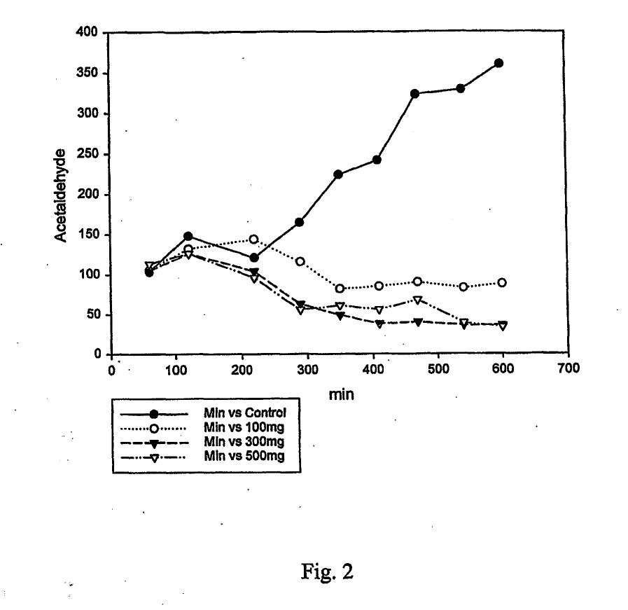

FIG. 2 shows the acetaldehyde content (μmol/l) of the contents of a human bowel as the function of time in a control sample and in three samples, to which a preparation containing 100, 300, and 500 mg of an acetaldehyde-binding substance was added.

“The acetaldehyde-binding substance” refers to a compound comprising one or more free sulphhydryl, amino or hydroxyl groups.

Cysteine and its derivatives are especially well suited to the purpose according to the invention. The most suitable amino acids for the use according to the invention are L and D-cysteines.

“The binding of acetaldehyde” refers to a chemical reaction between the acetaldehyde and the compound that has a free sulphhydryl and/or amino group, wherein the acetaldehyde jointly with the “acetaldehyde-binding substance” forms a larger molecule, and water can be formed in the reaction. For example, when reacting with cysteine, the acetaldehyde binds itself both to the sulphhydryl and the amino group and forms 2-methyl-L-thiazolidine-4-carboxylic acid and water. The acetaldehyde can bind itself to the amino group of almost any protein, whereby Schiff's base or a 2-methyl-imidazole ring is formed.

According to the invention, the compounds obtained from acetaldehyde by chemically binding are safe for the organism.

Suitable compounds for binding acetaldehyde in the organism also include the compounds according to the formula (I):

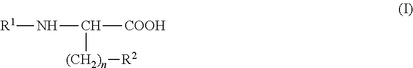

wherein
R¹ is hydrogen or an acyl group with 1-4 carbon atoms,
R² is a sulphhydryl or sulphone group,
n is 1 or 2.

The substances according to the following formula are capable of binding acetaldehyde:

R—NH₂ (II)

wherein R is derived from a protein (e.g., haemoglobin, albumin or tubuline)

In a reaction of the compound according to the formula (II) with acetaldehyde, a Shiff's base according to the formula (III) is formed,

R—N═CHCH₃ (III)

wherein
R is derived from a protein (e.g., haemoglobin, albumin or tubuline).

Amino acids or other compounds that suitably bind acetaldehyde and comprise a free sulphhydryl (SH) and/or amino (NH₂) group include:

### L-cysteine,

### D-cysteine,

Cysteic acid,
Cysteine glycine,
Threo or erythro-β-phenyl-DL-cysteine,
β-tetramethylene-DL-cysteine,

### Methionine,

D-penicillamine and its dipeptides with N-terminals,

### Semicarbazide,

Reduced glutathione,
β-mercaptoethylamine,

### D,L-homocysteine,

### N-acetylcysteine,

### L-cysteinyl-L-valine,

β-β-tetramethylene-DL-cysteine,

### Cysteinyl-glycine,

### Mercaptoethylglycine,

Tre-(5)-β-phenyl-DL-cysteine,

### Erythro-beta-phenyl-DL-cysteine,

### Thiaminhydrochloride,

### Sodiummetabisulphite,

### Arginine,

### Glycine,

### Lycine,

### Mercaptanes.

However, only such acetaldehyde-binding compounds that cause no health hazard in the amounts according to the invention are suitable for the preparations according to the present invention.

“The long-term binding of acetaldehyde” means keeping the acetaldehyde content for at least 30 minutes, preferably over 60 minutes, and most preferably over 120 minutes below a limit that is considered harmful, or preferably on a lower level than in a case where no pharmaceutical composition is used.

“A harmful/carcinogenic content of acetaldehyde” in the human mouth, oesophagus, stomach, and large intestine is 20-800 μmol/l of saliva or the contents of the intestine.

Keeping the acetaldehyde content essentially lower than without the use of the pharmaceutical composition means keeping the acetaldehyde content at a level that is at least 20%, preferably over 40%, and most preferably over 60% lower than when not using the pharmaceutical composition.

Such a harmful or carcinogenic content of acetaldehyde in the human mouth, oesophagus, stomach or large intestine can be obtained in connection with consuming alcoholic drinks, particularly strong alcoholic drinks, or foodstuffs containing alcohol, as a consequence of smoking or when consuming preparations containing acetaldehyde.

“Alcoholic drinks” are ethanol-containing drinks, the ethanol content varying within 0.7% by volume and 84% by volume.”

“Alcoholic foodstuffs” refer to foodstuffs containing at least 0.7% of ethanol. Such foodstuffs can be, for example, fermented juices or preserves, or foodstuffs preserved with small amounts of alcohol, pastries, jellies, and mousse seasoned with liqueur or corresponding preparations containing alcohol.

The use of the preparations according to the invention can be of benefit even, when light alcoholic drinks or foodstuffs are consumed, which contain small amounts of alcohol. Some foodstuffs can also already contain acetaldehyde. Acetaldehyde-containing foodstuffs, which have ethanol that is generated in connection with fermentation, such as beer, cider, wine, home-brewed beer, and other alcoholic drinks, as well as many juices. As for alcoholic drinks, sherry contains an especially large amount of acetaldehyde.

“In connection with consuming alcohol” herein refers to the period of time that begins from starting to enjoy alcohol and ends, when there is no more alcohol in the blood.

“In connection with smoking” herein refers to the period of time that begins from starting to smoke and ends, when smoking is stopped.

### Pharmaceutical Preparation Affecting the Mouth

“A local, long-acting preparation that is placed in the mouth” refers to all preparations that are placed between the cheek or the lip and the gum, or preparations that are sucked or chewed in the mouth, and in which the release of the substance intended to have a local effect in the mouth, the pharynx, the oesophagus or the stomach is prolonged.

“A prolonged release of the effective substance” means that the release of the substance takes 30 minutes at the minimum, preferably 120 minutes at the minimum, most preferably over four hours. By using the compositions according to the invention, release times of the effective substance of as much as 4-8 hours can be achieved.

The compounds that are used in the preparation that binds acetaldehyde can be compounds comprising one or more free sulphhydryl and/or amino groups.

In addition to the acetaldehyde-binding, so-called effective substance(s), at least one substance that regulates the release rate of the effective substance is added to the locally long-acting pharmaceutical composition, which is placed in the mouth and can be in the form of a tablet. It is preferably that the composition also ensures that the preparation adheres to the mucous membrane of the mouth. For these purposes, mostly two polymers are used, such as cellulose derivatives, chitosans, alginates, polyethylene glycols, carbomers or polycarbophils, preferably HPMC derivatives and carbomers, and most preferably a mixture of a HPMC-quality Methocel K4M and a carbomer-quality Carbopol 971, which are generally used as pharmaceutical additives, are known to be safe, and which in the physiological conditions of the mouth form a gel. With the aid of the substances used, both the release rate of the effective substance can advantageously be regulated and the adherence of the preparation to the mucous membrane of the mouth can be ensured. By varying the molecular size and the amount of polymers and, when using mixtures of various polymers, their mutual relationships, the release rate of the effective substance and the adhesion of the preparation to the mucous membrane can be regulated.

The total amount of polymers in the preparation is 10-50%, preferably 15-40%, and most preferably 20-30%.

A dosage unit of the pharmaceutical composition can comprise 50-500 mg of acetaldehyde-binding substance; preferably the amount of acetaldehyde-binding substance is 50-300 mg, and most preferably 100-200 mg.

In the oral conditions, preferably 15-25 mg of the compound is released in an hour.

The preparations according to the invention can be placed in the mouth 1 or 2 at a time and they can be replaced by new ones at 4 to 10-hour intervals, preferably at 6 to 8-hour intervals.

The composition of the long-acting tablet that is placed in the mouth can be as follows, for example:

| | |
|---|---|
| Acetaldehyde-binding substances | 100-200 mg |
| Non-ionised macromolecules | 20-50 mg |
| Ionising polymers | 6-10 mg |
| Lubricants | 1-3 mg |

Non-ionised macromolecules include, for example, methylcellulose (MC), hydroxypropylcellulose (HPC), and hydroxypropyl-methylcellulose (HPMC), and polyethylene glycol (PEG). Ionising polymers include, for example, sodium carboxy-methyl cellulose (NaCMC), alginic acid, sodium alginate, chitosan, polycarbophil (Noveon™), and cabomer (Carbopol™)

### Pharmaceutical Preparation Affecting the Stomach

“A long-acting preparation that has a local effect on the stomach” refers to all monolithic or multiparticular tablets or capsules or granules as such, which, when wetted under the influence of the gastric juices adhere to the mucous membrane of the stomach or form a gel that floats in the contents of the stomach, as a consequence of which their residence time in the stomach is prolonged and thus enables a prolonged release in and a local effect of the drug on the stomach. The long-acting preparation that locally acts on the stomach can be a liquid preparation taken orally (mixture), the physical structure of which is a gel.

A special property required of the pharmaceutical composition that has a local effect on the stomach is that it remains in the stomach for as long as possible. Technically, this can be solved in two ways: by making a preparation that adheres to the mucous membrane of the stomach or making a preparation that floats in the contents of the stomach. The preparation can be rendered fixable to the mucous membrane of the stomach by using as additives cationic polymers, such as various chitosan grades. Preparations that float in the stomach are provided by using polymers (e.g., alginic acid) that form a gel and by adding to the preparation sodium hydrogen carbonate, which under the influence of gastric acid releases carbon dioxide, which in turn forms gas bubbles inside the gel. A liquid gel that floats in the stomach can also be prepared from sodium alginate, aluminium hydroxide, sodium hydrogen carbonate, and water, to which the acetaldehyde-binding compound can be added. A corresponding liquid preparation is also obtained by adding an acetaldehyde-binding substance to an aqueous dispersion of chitosan. Another preparation that remains in the stomach for a long time is a preparation, which is known as HBS™ (hydrodynamically balanced system). The preparation can remain in the stomach for a long time, when a relatively large tablet is made of it (with a diameter of at least 7-10 mm) and it is coated with a film, which does not decompose in the alimentary tract, and which, however, releases an effective substance (Oros™) through a hole which has been made to it, for example. However, a prerequisite is that such a preparation be consumed after eating.

A single dose of the pharmaceutical composition having a local effect on the stomach comprises 50-500 mg of acetaldehyde-binding substance; preferably the amount of acetaldehyde-binding substance is 50-300 mg, and most preferably 100-200 mg.

When needed, the dosage is renewed at 4 to 10-hour intervals, preferably at 6 to 8-hour intervals.

The amount of compound released in the conditions of the stomach is preferably 40-80 mg in an hour.

The preparation according to the invention, which releases in the stomach, has at least one—often two—polymers, which have the task of keeping the drug as long as possible, for two hours minimum, in the stomach either so that it attaches the preparation to the mucous membrane of the stomach or forms a gel that floats in the contents of the stomach. Another task of the polymers is to prolong the release of the effective substance.

The preparation that locally binds acetaldehyde in the stomach can be a tablet that forms a gel in the stomach or a capsule comprising a mixture of powder or granules that forms a gel. In addition to the acetaldehyde-binding substances, the preparation comprises polymers that form a gel in the stomach, such as chitosans, alginates, sodium carboxy-methylcellulose grades, carbomers or aluminium hydroxide. To advance floating in the stomach, the preparation can also comprise sodium hydrogen carbonate.

The amount of polymers in the preparation is 10-50%, preferably 15-40%, and most preferably 20-30%.

The amount of sodium hydrogen carbonate can be 10-30%, preferably 20% of the amount of polymers.

The preparation that locally binds acetaldehyde in the stomach can be a tablet or granule preparation, wherein the acetaldehyde-binding substance is mixed with the fillers needed and, after that, granulated by using enteric polymers as binders. The binder used can be any known enteric polymer, preferably a polymer with a solution pH of 6-7, and most preferably the polymer is any of the methacrylate derivatives, which are known by the trade names Eudragit L and Eudragit S. The amount of enteric polymer in the preparation is preferably 2-5%, most preferably 3-4%.

The preparation that locally binds acetaldehyde in the stomach can be a liquid preparation, i.e., a mixture comprising, in addition to the acetaldehyde-binding substance, also sodium alginate, aluminium hydroxide, sodium hydrogen carbonate, and water. The amount of water in the whole preparation is 70-90%, most preferably about 75-85%. The amount of sodium alginate in the preparation is preferably 2-10%, most preferably about 5%, and the amount of aluminium hydroxide is preferably 5-15%, most preferably about 10%.

The relative composition of the preparation comprising granules can be as follows, for example:

| | |
|---|---|
| Acetaldehyde-binding substances | 60 parts |
| Chitosan | 10-40 parts |
| Calcium hydrogen phosphate | 0-30 parts |

The relative composition of the liquid preparation can be as follows, for example:

| | |
|---|---|
| Acetaldehyde-binding substances | 10 parts |
| Sodium alginate | 2-10 parts |
| Aluminium hydroxide | 5-15 parts |
| Sodium hydrogen carbonate | 1-2 parts |
| Water | 70-80 parts |

### Pharmaceutical Preparation Affecting the Large Intestine

“A long-acting preparation that has a local effect in the large intestine” refers to all monolithic or multiparticular tablets or capsules or granules as such, which will not release the dose in a prolonged way until the preparation has drifted to the end of the small intestine or all the way to the large intestine.

The preparation according to the invention that releases acetaldehyde-binding substances in the large intestine in a prolonged way, carries the acetaldehyde-binding substance to the last part of the small intestine or to the large intestine before the substance in question is allowed to be released—whichever the releasing mechanism.

The pharmaceutical composition that binds acetaldehyde in the large intestine is administered orally. There are numerous techniques available for directing the release of an orally dosed drug to the large intestine. The most functional solutions are based on the use of enteric polymers. A film coating, which does not dissolve in the acidic environment of the stomach, but dissolves at a pH value of 7 at the latest, can be made both on the tablet and the granules. In making the preparation, it is also possible to use polysaccharides that degrade under the effect of microbes of the large intestine, or polymers generated by azo bonds. The form of preparation known by the trade name Oros™ can also be used, when its opening is first covered with an enteric polymer, the solution pH of which is ≈7.

Useful enteric polymers include, for example, the grades of hydroxypropyl methylcellulose-acetatesuccinate (HPMC-AS) sold by the trade name Agoat™, Aqoat AS-HF™ in particular, a cellulose acetatephtalate (CAP) grade sold by the trade name Aquateric™, and methacrylic acid-methylmethacrylate copolymers, the grade sold by the trade name Eudragit-S™ in particular.

The preparation according to the invention has at least one ingredient, which adjusts the release of the effective substance not to take place until at the end of the small intestine or in the large intestine. This component can be a polymer that dissolves depending on the pH (=enteric polymer) or a polymer that degrades under the effect of the enzymes secreted by the bacteria of the large intestine. The polymer that controls the place of release can form a film around the entire preparation. It can also form a film around the particles (granules) contained by the multiple-part preparation. The polymer that degrades under the effect of the enzymes secreted by the bacteria of the large intestine can also be as a filler in a monolithic preparation, or as a filler in the granules or in a multiple-unit preparation prepared from these granules.

The preparation according to the invention is an enteric tablet, the film coating of which does not dissolve until at the end of the small intestine or at the beginning of the large intestine. The dissolution pH of the polymer that forms the film is 6.0-7.5, preferably 6.5-7.0. The amount of enteric polymer that forms the film is 5-20%, preferably 10-15% of the whole mass of the tablet. The filler of the tablet can comprise pharmaceutical additives that do not swell, such as calcium hydrogen phosphate.

The preparation according to the invention can also be granules that comprise an acetaldehyde-binding substance and are coated with an enteric film, the dissolution pH of the film-forming polymer being 6.0-7.5, preferably 6.5-7.0. The amount of film-forming enteric polymer of the entire mass of the granule is 5-30%, preferably 15-25%. The granule can comprise 20-40%, preferably about 30% of filler poorly soluble in water, such as calcium hydrogen phosphate.

The binder of the granule coated with the enteric film, according to the invention, can be an enteric polymer, the dissolution pH of which is 6.0-7.5, preferably 6.5-7.0. The amount of binder in the granule is 2-5%, preferably 3-4%.

The preparation according to the invention can also be a tablet comprising the enteric coated granules described above, on which an enteric film has also been made. The tablet made for such a preparation not only comprises enteric granules, but also a filler suitable for direct compression, such as microcrystalline cellulose, the amount of which in the tablet is 30-70%, preferably 40-60%.

The dosage unit of the pharmaceutical composition preferably comprises 50-500 mg of acetaldehyde-binding substance; preferably the amount of acetaldehyde-binding substance is 50-300 mg, and most preferably 100-200 mg.

The amount of compound releasing in the conditions of the large intestine is preferably 50-100 mg in an hour.

When needed, the dosage can be repeated at 4 to 10-hour intervals, preferably at 6 to 8-hour intervals.

The composition of the enteric tablet, which comprises enteric granules and binds acetaldehyde in the desired way, can be as follows, for example:

| | | | |
|---|---|---|---|
| Enteric granules: | Acetaldehyde-binding substance | 100 | mg |
| | Filler, e.g., calcium hydrogen phosphate | 30-50 | mg |
| | Enteric polymers | 40-60 | mg |
| Enteric tablet: | Enteric granules | 170-210 | mg |
| | Microcrystalline cellulose | 170-210 | mg |
| | Lubricants (e.g. magnesium stearate | 5-10 | mg |
| | and talcum) | | |
| | Enteric polymers | 30-50 | mg |

### Administration of Acetaldehyde-Binding Compositions

The content of acetaldehyde formed in saliva as a consequence of consuming alcoholic drinks, smoking or for some other reason can be decreased so that, for example, in connection with consuming alcoholic drinks or smoking, a preparation is placed in the mouth, under the upper lip, for example, which at a suitable rate releases cysteine or a similar acetaldehyde binding agents. In this case, the acetaldehyde content of saliva decreases by over 20%, preferably by over 40%, most preferably over 60%, typically by 60-80% compared with a placebo. For example, 100 mg of cysteine in the preparation is enough to have the desired effect for 4-5 hours. When needed, a new preparation is placed in the mouth after the previous one has dissolved. This is repeated as long as there is alcohol in the blood.

Similarly, the acetaldehyde content locally increased in the stomach as a consequence of consuming alcoholic drinks, for example, can be decreased by more than 20%, preferably over 40%, most preferably over 60%, typically 60-80% compared with a placebo by consuming, in connection with alcoholic drinks, a pharmaceutical composition that releases an acetaldehyde-binding compound at a suitable rate in the stomach.

According to the invention, the preparations that bind acetaldehyde and affect the mouth, the stomach, and the large intestine can also be used simultaneously.

The acetaldehyde content formed from consumed or endogenous ethanol in the large intestine can be decreased by over 20%, preferably over 40%, most preferably 60-80% compared with a placebo by consuming a preparation, which in the large intestine releases acetaldehyde-binding compounds at a suitable rate.

In the following, the invention is examined with the aid of examples.

### EXAMPLE 1

Immediately before consuming an alcoholic drink, either a placebo or a preparation that slowly released cysteine at a suitable rate was attached to the gums under the upper lips of nine participants in a test. The amount of cysteine in the preparation was 100 mg. During 20 minutes, the participants consumed 0.8 g/kg of ethanol in the form of a drink containing 10% by volume of ethanol. During the next 320 minutes, the acetaldehyde contents of the saliva of the testees were measured at 20-minute intervals. The acetaldehyde contents of the saliva of the participants who used the cysteine-containing preparation was 3-5 times lower than that of the participants in the reference group during the entire measuring period. After consumption of alcohol, approximately 66% of the carcinogenic acetaldehyde could be removed by using an acetaldehyde-binding pharmaceutical composition. FIG. 1 shows the acetaldehyde content (Ach μmol/l) measured for the saliva of the control group testees () and the group, who used the preparation according to the invention (◯), as a function of time (min).

### EXAMPLE 2

The pharmaceutical composition placed in the mouth, which has a long-term local effect, can be prepared and used to decrease the risk of cancer caused by acetaldehyde as follows:

The composition of a capsule placed in the mouth can be as follows, for example:

| | |
|---|---|
| Cysteine | 100.0 mg |
| HPMC (Methocel K4M ™) | 30.0 mg |
| Carbomer (Carbopol 971P NF ™ | 6.9 mg |
| Magnesium stearate | 1.4 mg |

Cysteine, HPMC, and carbomer are mixed carefully by using mixers generally used in the pharmaceutical industry. At the final stage, magnesium stearate is also added to the mixture to function as lubricant of the mould of the tablet-compressing machine. Tablets (with a diameter of 9 mm) are compressed from the powder mixture by using conventional tablet machines.

The preparation is placed in the mouth in connection with consuming alcohol. As long as there is alcohol in the blood, a new capsule is taken after the previous one has dissolved.

### EXAMPLE 3

The locally long-acting pharmaceutical preparation that binds acetaldehyde in the stomach can be prepared and used to decrease the risk of cancer caused by acetaldehyde as follows:

The relative composition of the preparation that locally binds acetaldehyde in the stomach can be as follows, for example:

| | |
|---|---|
| Cysteine | 60 parts |
| Chitosan | 10-40 parts |
| Calcium hydrogen phosphate | 0-30 parts |

The powder mixture is mixed by conventional mixers (such as a blender), which are used in the drug industry. After that, the powder mixture is granulated using a 2.5% acetic acid as a granulation liquid. The granulation liquid can be added to the same blender. The moist powder mass is compresses through a screen plate or a perforated plate (the diameter of the aperture being 2 mm). The formed granules are dried and screened. A screen fraction of 1.2-1.7 mm is recovered, which is dispensed into hard gelatine capsules so that the dose of cysteine is 100 mg.

The tablets prepared above are ingested to decrease the risk of cancer locally caused by acetaldehyde in occasions, which are favourable for an increase in the acetaldehyde content of the stomach, such as in connection with consuming alcoholic drinks. The dosage is given at 4 to 6-hour intervals as long as there is alcohol in the blood.

### EXAMPLE 4

The pharmaceutical composition that releases acetaldehyde-binding substances in the large intestine in a prolonged way can be prepared and used to decrease the risk of cancer caused by acetaldehyde as follows.

The composition of the enteric tablet, which comprises enteric granules and binds acetaldehyde in the desired way, can be as follows, for example:

| | | |
|---|---|---|
| Enteric granules: | Cysteine | 100 mg |
| | Calcium hydrogen phosphate | 40 mg |
| | Eudragit-S | 5 mg |
| | Aqoat AS-HF | 40 mg |
| Enteric tablet: | Enteric granules | 185 mg |
| | Microcrystalline cellulose | 185 mg |
| | Magnesium stearate | 4 mg |
| | Talcum | 4 mg |
| | Aqoat AS-HF | 40 mg |

Cysteine and the calcium hydrogen phosphate that works as a filling agent are mixed together. Eudragit S is dissolved in ethanol (a 20% solution) and the solution is used to moisten the powder mixture. The wet mass is compressed into granules. The dried granules are screened and a granule fraction of 1.2-1.7 mm is coated with Aqoat AS-HF. The composition of the coating solution is as follows: Aqoat AS-HF 10%, triethylcitrate 3.5%, magnesium strearate 3%, and water 83.5%. The coated granules are mixed with microcrystalline cellulose (e.g., Emcocel LP200™) and, finally, the lubricants are added to the mixture: magnesium stearate and talcum. Next, the mixture is compressed into tablets and, finally, an enteric film is made on the tablet in the same way as on the granules. In all stages of operation, mixers, granulators, screening equipment, film coating equipment, and tablet compressing machines, which are generally used in the pharmaceutical industry, can be used.

The composition prepared above is ingested orally in connection with consuming alcoholic drinks and the dosage is repeated at 4 to 6-hour intervals as long as there is alcohol in the blood.

### EXAMPLE 5

In the trial, acetaldehyde was inactivated in vitro in the contents of the intestine by using the tablet according to the invention, which slowly released cysteine. The preparation was a compression-coated tablet, the coating material thereof being a polysaccharide, pectin, which degrades under the influence of microbes of the large intestine. The composition of the preparation used in the trial was as follows:

### L-cysteine 100 mg

### Pectin 190 mg

Microcrystalline cellulose 50 mg
Hydroxypropyl methylcellulose 100 mg
Polyvinyl pyrrolidone 42 mg

### Talcum 2 mg

Magnesium stearate 2 mg

Two volunteers participated in the trial, and the contents of their intestines were collected with the aid of Colonsteril intestine evacuation medicine. The obtained contents of the intestines (the excrement) were diluted with distilled water in a ratio of 1:1 to convert the excrement into a form, which is easier to process and is more soluble.

After this, the contents of the intestine were divided into four parts (1-4) 50 ml each. Ethanol was added to the samples, its final concentration thus being 35 mM in each sample.

1st sample=control without cysteine (contained additives of tablets, e.g., pectin)
2nd sample=100 mg of cysteine (i.e., one tablet)
3rd sample=300 mg of cysteine (i.e., 3 tablets)
4th sample=500 mg of cysteine (i.e., 5 tablets)

The samples were incubated (i.e., the conditions in question were maintained to cause a reaction) at a temperature of 37° C. in a water bath (corresponding to the temperature of the human body), slightly mixing all the time.

500-μl samples were taken from the excrement samples at about 1-hour intervals to analyse the concentrations of acetaldehyde and ethanol by gas chromatography. These samples were taken during a period of 600 minutes in total.

FIG. 2 shows the acetaldehyde content of the intestine (μmol/l) as the function of time in a control sample, to which no preparation according to the invention had been added, and in three samples, to which preparations containing 100, 300, and 500 mg of effective substance had been added. A 100-mg cysteine tablet decreased the amount of acetaldehyde in the samples by 51% compared with the control sample and, 300-mg and 500-mg tablets by 68% and 66%, correspondingly.

Thus, the response of the acetaldehyde content of the samples is a good indication of how the preparation according to the invention considerably decreases the acetaldehyde content generated from ethanol under the effect of microbes in the contents of the intestine. The conditions corresponded well to in vivo conditions.

It is especially surprising that the effective substance, which contains a very reactive SH group, reacts in the intestinal juice exactly as desired, i.e., the effective substance, for example, is not uselessly consumed in reactions with the other compounds of the contents of the intestine.

### EXAMPLE 6

### Preparation of the Capsules

The capsules were prepared by mixing 500 g of L-cysteine (Gonmisol S. A., Spain), 500 g of Eudragit RS-PO, forming a matrix structure (Evonik Röhm GmbH, Germany), and 1 kg of calcium hydrogen phosphate (Emcompress® Anhydrous; Mendell a Penwest Company, Lakeville, Minn.) in a Turbula Powder Blender (Glen Mills Inc., Clifton, N.J.) for 10 minutes.

The mixture was wet-granulated using ethanol. The obtained wet granules were sieved using a 2-mm sieve, and thereafter allowed to dry at room temperature in a fume hood for 24 hours. The dried granules were sieved using a 1.68 mm and 1.18 mm sieves, and the obtained middle fraction was collected for capsulation.

Simultaneously, a placebo formulation, where the L-cysteine was replaced by the same amount of CaHPO₄, was prepared following the exact same procedure.

The obtained matrix granules were weighed into HPMC capsules so that the desired amount of cysteine per capsule was obtained. The L-cysteine concentration of the granules was determined using a capillary method (400 mg of granules contained 98 mg of L-cysteine). The amount of L-cysteine per capsule was left at 50 mg in order to ease the selection of a suitable dosage (for a dosage of 100 mg or 200 mg of L-cysteine, 2 or 4 capsules were administered at essentially the same time to the subject).

Similar capsules containing also titanium dioxide were prepared, and this excipient was found not to have an effect on the desired function of the capsule.

The capsules prepared above are ingested to decrease the risk of cancer locally caused by acetaldehyde in occasions, which are favourable for an increase in the acetaldehyde content of the stomach, such as in connection with consuming alcoholic drinks. The dosage is given at 4 to 6-hour intervals as long as there is alcohol in the blood.

### EXAMPLE 7

### Dissolution Test for the Capsules

Dissolution tests were carried out on the capsules of Example 6 according to the USP I method (USP 24) (The United States Pharmacopeia 2001). A standard curve was prepared between 0.01 and 0.6 mg/ml (y=2.196+0.0016, r²=0.9999). The medium used was 500 ml of pH 1.2HCl buffer. The rotation rate of the baskets was 100 rpm, and the temperature of the medium was +37° C. (±0.5). Samples were taken at 5-minute intervals for the first half hour and thereafter at 10-minute intervals for the remaining 2 hours. L-cysteine was detected in flow-through cells (10 mm) at a wavelength of 213 nm. The results were calculated by using dissolution software. The system was equipped with a bath and a pump (Sotax AT7 UV Dissolution System, Stax, Allschwil, Switzerland) and a spectrophotometer (PerkinElmer, Lambda 25, PerkinElmer, Inc., Waltham, Mass.), the software used for the test and for calculating the results was WinSotax (Sotax).

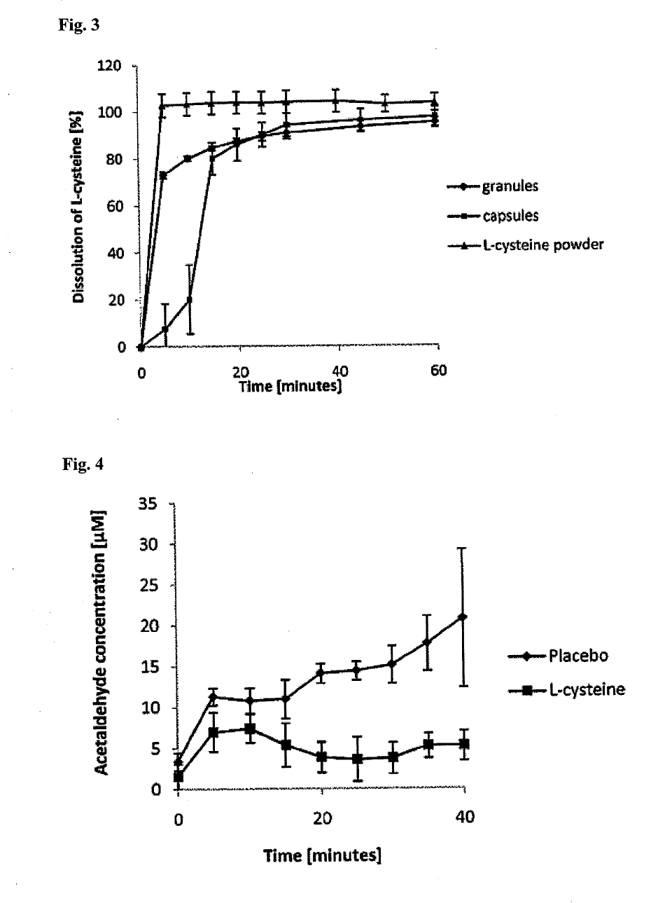

This dissolution test showed that the formulation released L-cysteine at a controlled rate, yet fast enough to have time to react with acetaldehyde before leaving the stomach. These results are shown in FIG. 3. When not granulated, the L-cysteine was dissolved rapidly (100% in 5 minutes).

### EXAMPLE 8

### Acetaldehyde-Binding

### Study Procedure:

Seven volunteers (2 men, 5 women) with achlorhydric atrophic gastritis participated in the study. Their mean age±SD was 57±7 years and mean body weight 75±22 kg. All volunteers were non-smokers and normal social drinkers, with an average consumption of 50 g or less of ethanol per week.

A randomized double-blinded placebo-controlled study design was used, and each participant served as his/her own control. The 2 study days were separated by at least a 3-day interval. The volunteers were told to refrain from alcohol intake for 24 hours and food intake for 12 hours prior to the study.

A nasogastric tube (Duodenal tube Levin, CH10, Unomedical, Denmark) was inserted into the subjects to a depth of 55 cm at the beginning of each study day. The tube was lubricated with Xylocaln gel (AstraZeneca, Södertälje, Sweden) containing no ethanol. During the tube placement, the volunteers were given 100 ml of water to facilitate swallowing of the tube.

The subjects were given four capsules, containing either cysteine (50 mg in each capsule) or placebo, as prepared according to Example 6, orally double blindly with 200 ml of water. Immediately thereafter, ethanol (0.3 g/kg body weight) diluted in water to 15 vol %, was infused via the nasogastric tube into the stomach of the volunteers.

Samples of gastric juice (5 ml) were aspirated through the tube at 5-minute intervals up to 60 minutes after the ethanol infusion or until the stomach had emptied, as indicated by unsuccessful aspiration. The samples were analyzed for pH and acetaldehyde, ethanol and cysteine concentrations.

### Analysis:

To measure the acetaldehyde concentration, 450 μl of gastric juice was immediately transferred into a headspace vial containing 50 μl of 6 mol/l perchloric acid. Perchloric acid does not hydrolyze the cysteine-acetaldehyde bond.

For the ethanol analysis, the gastric juice was diluted 10-fold in purified water, and 500 μl of diluted gastric juice was transferred into a headspace vial.

Two parallel samples were used for the measurements, and the mean value was calculated. The levels of acetaldehyde and ethanol were analyzed by headspace gas chromatography, as previously described (Väkeväinen et al., 2002, Scand J Gastroenterol, 37:648-655).

L-cysteine concentration of the gastric juice samples were determined by using an HPLC method. A standard curve was prepared between concentrations of 0.0625 and 2.0 mg/ml (y=851.06x+8.52, r²=0.9704). Two parallel samples were again prepared. 60 μl of gastric juice was measured into a test tube, and 30 μl of pH 7.4 phosphate-buffered saline solution and 30 μl of 20 vol-% tri-n-butyl phosphine in dimethylformamide were added. The samples were incubated for 30 minutes at +4° C., after which 90 μl of cold 10% trichloroacetic acid containing 1 mM Na₂EDTA was added, and the samples were vortexed for 2 minutes and then centrifuged for 10 minutes at 4500 rpm. 50 μl of supernatant was pipette into a test tube containing 125 μl of pH 9.5 borate buffer with 4 mM Na₂EDTA, 10 μl of 1.55 M sodium hydroxide, and 50 μl of 2 mg/ml 4-fluoro-7-sulfobenzofurazan, ammonium salt (SBD-F) solution in borate buffer. The samples were incubated for 60 minutes at +60° C. so that a yellow derivate was formed. Thereafter, 150 μl of the solution was pipette into HPLC inserts, and used for the measurements. The isocratic mobile phase was pH 7.0 phosphate buffer and methanol (95:5). The flow rate was 1 ml/min and the retention time was 6 minutes. The L-cysteine concentration was determined using a fluorescence detector (excitation 385 nm, emission 515 nm).

### Results:

FIG. 4 shows the effect of the L-cysteine administration (or the placebo administration) on the acetaldehyde levels. In all measurements, the average acetaldehyde concentration of the gastric juice was 2.6 times higher with placebo than with cysteine. No significant differences existed in ethanol concentrations between cysteine and placebo treatments. The average ethanol concentration in the gastric juice was 5.0 vol-% in the first sample, declining to 0.9 vol-% in the 40-minute sample. A positive correlation emerged between the acetaldehyde concentration and the ethanol concentration.

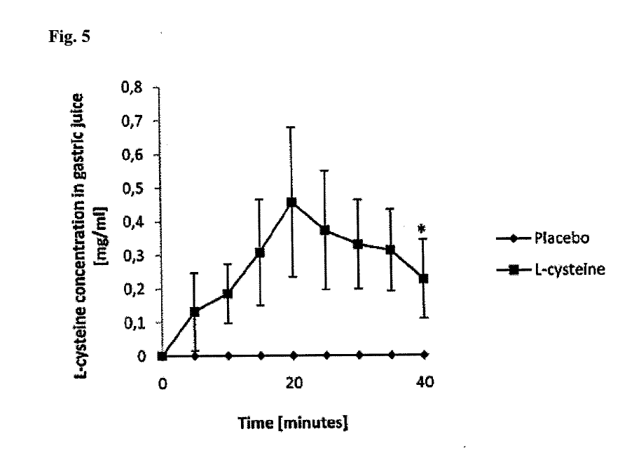

L-cysteine was detected in the gastric juice of all volunteers after the administration of study formulations containing L-cysteine. The mean cysteine concentrations are represented in FIG. 5. After administration of placebo formulations, no L-cysteine was detected. No significant correlation was found between the cysteine concentration and the acetaldehyde concentration.

1. The use of compounds, which bind acetaldehyde and comprise one or more free sulphhydryl and/or amino groups, for preparing a pharmaceutical composition for locally binding acetaldehyde in the long term in saliva, the stomach or the large intestine so that the acetaldehyde-binding compound is bound to such a pharmaceutically acceptable carrier, which regulates the release rate of the compound in the mouth, the stomach or the large intestine to maintain the acetaldehyde content below a limit that is considered harmful or essentially lower than without the use of the pharmaceutical composition.

2. The use according to claim 1, characterized in that the pharmaceutical composition comprises one or more substances from the group: L-cysteine, D-cysteine, cysteic acid, cysteine glycine, threo- or erythro-β-phenyl-DL-cysteine, β-tetramethylene-DL-cysteine, L-ascorbic acid, D-penicillamine and its N-terminal dipeptides, semicarbazide, reduced glutathione, D,L-homocysteine, N-acetylcysteine, L-cysteinyl-L-valine, β-β-tetramethylene-DL-cysteine, cysteinyl glycine, mercaptoethyl glycine, tre-(5)-β-phenyl-DL-cysteine, erythro-β-phenyl-DL-cysteine, thiamine hydrochloride, sodium metabisulphite, arginine, glycine, and lycine.

3. The use according to claim 1, characterized in that the pharmaceutical composition comprises one or more compounds according to the formula (I), 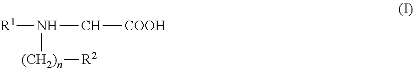
wherein
R¹ is hydrogen or an acyl group containing 1-4 carbon atoms,
R² is a sulphhydryl or sulphone group and
n is 1 or 2.

4. The use according to claim 1, characterized in that the pharmaceutical composition comprises an acetaldehyde-binding amino acid, to which the acetaldehyde binds itself both through a sulphhydryl and an amino group.

5. The use according to claim 1, characterized in that the pharmaceutical composition comprises one or more compounds according to the formula (II)
R—NH₂ (II)
wherein R is derived from a protein (e.g., haemoglobin, albumin or tubulin).

6. The use according to claim 1, characterized in that the carrier of the pharmaceutical composition is selected so that the composition is capable of selectively releasing the said acetaldehyde-binding compound(s) in the conditions of the mouth, the stomach or the large intestine only.

7. A pharmaceutical composition for binding acetaldehyde from saliva, characterized in that
the composition comprises one or more substances, which bind acetaldehyde and are bound to a pharmaceutically acceptable carrier, comprising one or more free sulphhydryl and/or amino groups, and
the carrier controls the releasing speed of the effective substance so that the acetaldehyde-binding substances are released to saliva for at least 30 minutes in the conditions of the mouth.

8. The composition according to claim 7, characterized in that the composition comprises 50-500 mg, preferably 50-300 mg, and most preferably 100-200 mg of acetaldehyde-binding substance.

9. A composition according to claim 7, characterized in that the carrier of the composition comprises one or more substances, which are generally used as pharmaceutical additives and which form a gel in the physiological conditions of the mouth, and which are selected from the group: cellulose derivatives, chitosans, alginates, polyethylene glycols, carbomers, polycarbophils, preferably methylcellulose (MC), hydroxypropyl cellulose (HPC), and hydroxypropyl methylcellulose (HPMC), preferably derivatives of hydroxypropyl methylcellulose and carbomer derivatives.

10. The composition according to claim 9, characterized in that the total amount of polymers in the composition is 10-50%, preferably 15-40%, and most preferably 20-30% of the weight.

11. A pharmaceutical composition for binding acetaldehyde from the stomach, characterized in that
the composition comprises one or more substances, which bind acetaldehyde and comprise one or more free sulphhydryl and/or amino groups, and which are bound to a pharmaceutically acceptable carrier, and
the carrier controls the release rate of the effective substance so that the acetaldehyde-binding substances are released into the stomach for 0.5 to 8 hours.

12. The composition according to claim 11, characterized in that it comprises 50-500 mg, preferably 50-300 mg, and most preferably 100-200 mg of acetaldehyde-binding substance per single dose.

13. The composition according to claim 11, characterized in that the carrier of the composition comprises one or more substances, which are selected from the following group: various chitosans, alginates, such as sodium alginate, aluminium hydroxide, sodium hydrogen carbonate, sodium carboxymethyl cellulose, and sodium hydrogen carbonate.

14. The composition according to claim 13, characterized in that it comprises 10-30%, preferably 20% sodium hydrogen carbonate of the weight of the polymers.

15. The composition according to claim 11, characterized in that the total amount of polymers in the composition is 10-50%, preferably 15-40%, and most preferably 20-30% of the weight.

16. The composition according to claim 11, characterized in that it is granulated by using, as binders, enteric polymers, preferably methacrylate derivatives, the solution pH of which is 6-7 and the total amount of the weight of the preparation is 2-5%, preferably 3-4%.

17. The composition according to claim 11, characterized in that it is a liquid preparation comprising sodium alginate, aluminium hydroxide, and sodium hydrogen carbonate, comprising 70-90%, preferably 75-85% of water, preferably 2-10%, most preferably 5% of sodium alginate, and preferably 5-20%, most preferably 8-11% aluminium hydroxide of the weight of the composition.

18. A pharmaceutical composition for binding acetaldehyde from the large intestine, characterized in that
the composition comprises one or more substances, which are bound to a pharmaceutically acceptable carrier, bind acetaldehyde, and comprise one or more sulphhydryl and/or amino groups, and
the carrier controls the release rate of the effective substance so that it is not released until in the conditions of the large intestine for 0.5-8 hours.

19. The composition according to claim 18, characterized in that the composition comprises 50-500 mg, preferably 50-300 mg, and most preferably 100-200 mg of acetaldehyde-binding substance.

20. The composition according to claim 18, characterized in that the composition is a coated tablet, the coating thereof comprising enteric polymer, which can be hydrolysed in a solution with a pH value of 6.0-7.5, preferably 6.5-7.0, and the amount of which in the entire mass of the tablet is 5-20%, preferably 10-15%.

21. The composition according to claim 18, characterized in that non-swelling pharmaceutical additives, such as calcium hydrogen phosphate and/or microcrystalline cellulose, are used in the prepared tablet, their amount in the tablet being 30-70%, preferably 40-60%.

22. The composition according to claim 18, characterized in that it comprises granules, which are coated with an enteric film and comprise an acetaldehyde-binding substance, the film-forming enteric polymer of which granules can be hydrolysed in a solution with a pH value of 6.0-7.5, preferably 6.5-7.0, and the amount of enteric polymer in the entire mass of the granule is 5-30%, preferably 6.5-7.0, and which granules optionally comprise, as fillers, poorly soluble substances, such as calcium hydrogen phosphate 20-40%, preferably 25-35%, and as binders enteric polymers, which can be hydrolysed in a solution with a pH value of 6.0-7.5, preferably 6.5-7.0, and the amount of which in the granule is 2-5%, preferably 3-4%.

23. The pharmaceutical composition according to claim 6, characterized in that it comprises a compound that has one or more free sulphhydryl and/or amino groups.

24. The composition according to claim 18, characterized in that the carrier of the composition comprises one or more substances from the group: enteric polymers, such as hydroxypropyl methylcellulose-acetatesuccinate (HPMC-AS), celluloseacetatephtalate (CAP), methacrylic acid-methyl methacrylate-copolymers.

25. The pharmaceutical composition according to claim 6, characterized in that it comprises one or more substances from the group: L-cysteine, D-cysteine, cysteic acid, cysteine glycine, threo- or erythro-β-phenyl-DL-cysteine, β-tetramethylene-DL-cysteine, L-ascorbic acid, D-penicillamine and its N-terminal dipeptides, semicarbazide, reduced glutathione, D,L-homocysteine, N-acetylcysteine, L-cysteinyl-L-valine, β-β-tetramethylene-DL-cysteine, cysteinyl glycine, mercaptoethyl glycine, tre(5)-β-phenyl-DL-cysteine, erythro-β-phenyl-DL-cysteine, thiamine hydrochloride, sodium metabisulphite, arginine, glycine, and lycine.

26. The use of L and/or D-cysteines for preparing a pharmaceutical composition for locally binding acetaldehyde on a long-term basis in saliva, the stomach or the large intestine so that the cysteine is bound to a pharmaceutically acceptable carrier that controls the release rate of the compound in the mouth, the stomach or the large intestine to maintain the acetaldehyde content below a limit considered harmful or essentially lower than without the use of the pharmaceutical composition.

27. A pharmaceutical composition for binding acetaldehyde from saliva, characterized in that
the composition comprises L and/or D-cysteines, which are bound to a pharmaceutically acceptable carrier, a substance comprising one or more free sulphhydryl or amino groups, and
the carrier controls the release rate of the cysteine so that it is released to the saliva for at least 30 minutes in the conditions of the mouth.

28. A pharmaceutical composition for binding acetaldehyde from the stomach, characterized in that
the composition comprises L and/or D-cysteines bound to a pharmaceutically acceptable carrier, and
the carrier controls the release rate of the cysteine so that the acetaldehyde-binding substances are released into the stomach for 0.5-8 hours.

29. A pharmaceutical composition for binding acetaldehyde from the large intestine, characterized in that
the composition comprises L and/or D-cysteines bound to a pharmaceutically acceptable carrier, and
the carrier controls the release rate of the cysteine so that it is not released until in the conditions of the large intestine for 0.5-8 hours.

30. A method for decreasing the risk of contracting cancer of the mouth, the pharynx, the oesophagus, the stomach or the large intestine caused by acetaldehyde, characterized in that the acetaldehyde produced by microbes from ethanol in saliva, the stomach or the large intestine is locally bound, by using the pharmaceutical composition according to claim 5, into a harmless form by consuming the said pharmaceutical compositions in connection with consuming alcoholic drinks.

31. The method according to claim 30, characterized in that the said pharmaceutical compositions are taken as long as there is alcohol in the organism and/or in the blood.

32. The method according to claim 30, characterized in that one or more pharmaceutical compositions, which are prepared in the form of tablets and comprise acetaldehyde-binding substance, are placed in the mouth in connection with consuming alcohol and replaced with a new one at 4 to 10, preferably 6 to 8-hour intervals.

33. The method according to claim 30, characterized in that the one or more pharmaceutical compositions in the form of tablets, comprising acetaldehyde-binding substance, are swallowed at 4 to 10-hour intervals, preferably at 6 to 8-hour intervals.

34. A locally acting sustained release pharmaceutical preparation comprising a granulated composition encapsulated in a capsule made of hydroxylpropyl methyl cellulose (HPMC), the granulated composition containing, as its active ingredient L- or D-cysteine, further containing calcium hydrogen phosphate as a filler, Eudragit RS-PO as a binder, and optionally containing further fillers, binders or other additives.

35. The pharmaceutical preparation according to claim 34, wherein the cysteine is L-cysteine, and is present in an amount of 100-200 mg per administered dose.

36. The pharmaceutical preparation according to claim 34, further containing titanium dioxide as a further additive.

37. A method for decreasing the risk of contracting cancer of the stomach, and consequently the large intestine, caused by the presence of acetaldehyde in these areas, wherein the preparation according to claim 34 is administered to a subject in connection with the subject smoking or in connection with the subject consuming alcoholic drinks or consuming alcohol-containing foodstuffs.

38. The method according to claim 37, wherein the preparations are administered at an interval of 4-10 hours, for as long as there is ethanol in the blood, or in connection with smoking.
